(19) United States
(12) Patent Application Publication
(10) Pub. No.: US 2009/0228002 A1
(43) Pub. Date: Sep. 10, 2009

(54) ELECTROMAGNETIC ENERGY ASSISTED TISSUE PENETRATION DEVICE AND METHOD

(76) Inventors: Robert F. Rioux, Ashland, MA (US); David J. Sauvageau, Methuen, MA (US)

Correspondence Address: BURNS & LEVINSON, LLP, 125 SUMMER STREET, BOSTON, MA, 02110, US

(21) Appl. No.: 12/398,064

(22) Filed: Mar. 4, 2009

Related U.S. Application Data
(60) Provisional application No. 61/068,039, filed on Mar. 4, 2008.

(51) Int. Cl. A61B 18/18

(52) U.S. Cl. 606/33

(57) ABSTRACT

A device and a method for creating access and therapeutically closing an opening in a tissue.

### CROSS-REFERENCE TO A RELATED APPLICATION

The present application claims priority to U.S. provisional application Ser. No. 61/068,039, filed Mar. 4, 2008, entitled “Vessel remodeling and lumen modification.” The entire contents the aforementioned provisional application is incorporated herein by reference.

### BACKGROUND

Various surgical procedures are performed by medical specialists such as cardiologists and radiologists, utilizing percutaneous entry into blood vessels. Usually, to facilitate cardiovascular procedures, a small gauge needle is introduced through skin and into a target blood vessel, often the femoral artery. The needle forms a puncture through the blood vessel wall at the distal end of a tract that extends through the overlying tissue. A guide wire is then introduced through the bore of the needle, and the needle is withdrawn over the guide wire. An introducer sheath is then advanced over the guide wire; the sheath and guide wire are left in place to provide access during subsequent procedures. The sheath facilitates passage of a variety of diagnostics and therapeutic instruments and devices into the vessel and its tributaries. Illustrative diagnostic procedures include angiography, intravascular ultrasonic imaging, and the like. Exemplary interventional procedures include angioplasty, antherectomy, stent and graft placement, emobilization, and the like. After this procedure is completed, the catheters, guide wire, and introducer sheath are removed, and it is necessary to close the vascular puncture to provide hemostasis and allow healing.

The most common technique for achieving hemostasis is to apply hard pressure on the patient's skin in the region of the tissue tract and vascular puncture to form a blood clot. Initially, pressure is applied manually and subsequently is maintained through the use of mechanical clamps and other pressure-applying devices. While effective in most cases, the application of external pressure to the patient's skin presents a number of disadvantages. When applied manually, the procedure is time-consuming and requires the presence of a medical professional for thirty minutes or more. For both manual and mechanical pressure application, the procedure is uncomfortable for the patient and frequently requires the administration of analgesics to be tolerable. Moreover, the application of excessive pressure can occlude the underlying artery, resulting in ischemia and/or thrombosis. Even after hemostasis has apparently been achieved, the patient must remain immobile and under observation for hours to prevent dislodgement of the clot and to assure that bleeding from the puncture wound does not resume. Renewed bleeding through the tissue tract is not uncommon and can result in hematoma, pseudoaneurisms, and arteriovenous fistulas. Such complications may require blood transfusion, surgical intervention, or other corrective procedures. The risk of these complications increases with the use of larger sheath sizes, which are frequently necessary in interventional procedures, and when the patient is anticoagulated with heparin or other drugs.

In recent years, several hemostasis techniques have been proposed to address the problem of scaling vessel wall punctures following percutaneous transcatheter procedures. In some cases bioabsorbable, thrombogenic plugs comprising collagen and other materials are placed proximal to the vessel wall puncture site to stop bleeding. The larger hemostasis plug stimulates blood coagulation in the vessel puncture site, but blocks the catheter entry tract, making catheter reentry more difficult, if required. Other existing procedures require the use of small dissolvable disks or anchors that are placed in the vessel to block or clamp the puncture hole. However, any device remaining in the vessel lumen increases the risk of thrombus formation. Such a device also can detach and cause occlusion in a distal blood vessel, which would likely require major surgery to remove. Other existing procedures include using needles and sutures delivered through catheters to ligate the puncture. These suturing procedures require particular skill. Suture material left in the vessel may cause tissue irritation that prolongs the healing process. Yet another existing procedure requires a procoagulant to be injected into the puncture, with a balloon catheter blocking inside the vessel lumen. However, in some cases, the clotting agent may leak past the balloon into the vessel lumen and cause stenosis. Still other existing procedures require the use of laser or of radio-frequency (RF) energy that is transmitted through the blood vessel through a catheter to thermally fuse or weld the punctured tissue together. All of the above procedures require either introducing and leaving foreign objects in the patient's body, and/or inserting a tubular probe of large diameter into the tissue channel left by the catheter in order to seal the puncture.

There is a need for an improved procedure for sealing a puncture left in a blood vessels and tissue after tissue penetration.

### SUMMARY

The present invention via embodiments disclosed hereinafter and many other embodiments within the scope of the claims of this patent overcome the problems as set forth above and/or afford other related advantages. The current disclosure describes various embodiments for speedy healing and closure of the opening. It is an aim of the disclosed embodiments and many other embodiments within the scope of the claims of this patent to reduce bleeding resulting from tissue and vessel penetration and to expedite healing.

One aspect of the invention disclosed hereinafter is a device for penetrating tissue. This device includes a removable access member which has a distal end and a proximal end. The proximal end of the device can be connected to a source of electromagnetic energy. The device also includes a sheath which encompasses the access member in a way that allows connecting the proximal end of the access member to the source of electromagnetic energy. The device permits the energy to be transmitted to the tissue through the distal end of the access member in such a way that penetration of the tissue and withdrawal from the tissue are facilitated by the use of the electromagnetic energy.

One other aspect of the invention disclosed hereinafter is a method for penetrating a tissue in order to perform a medical procedure. The method includes creating an access tract through the tissue while applying electromagnetic energy with a device which includes a removable access member and a sheath encompassing the access member. The method further includes withdrawing the access member while leaving the sheath in place. A medical procedure is then performed, after which the sheath is withdrawn.

While the present invention deals with a minimally invasive opening and closing of a vessel, it is equally understood that the invention can also be used to penetrate trough other collagen containing tissue utilizing electromagnetic energy to minimize subsequent healing.

### BRIEF DESCRIPTION OF THE DRAWINGS

The present invention, including device, apparatus and method aspects, is illustratively shown and described in reference to the accompanying drawings, in which

FIG. 1 is an illustration of an embodiment of a device of the current invention, which includes a removable access member for conducting electromagnetic energy to tissue and a sheath;

FIG. 2a illustrates an assembled embodiment of the device with the sheath substantially encompassing the removable access member;

FIG. 2b shows an enlargement of the distal end of a assembled embodiment of the device;

FIG. 3a illustrates the use of the device in a needle like procedure to access the inside of a vessel;

FIG. 3b illustrates the use of the sheath, which is left if place after removing the access member, for accessing the inside of a vessel or tissue;

FIG. 4a demonstrates the use of an embodiment of the device of the present invention for closing the wound on the vessel;

FIG. 4b illustrates the shrinking of the wound on the vessel as electromagnetic energy is applied to the wound via the device;

FIG. 5 illustrates the use of a drop of saline held in place while withdrawing the device of the present invention from the vessel;

FIG. 6 shows a cross section of a bipolar embodiment of the access member having a hollow channel inside;

FIG. 7a shows a cross section of a monopolar embodiment of the access member utilizing a conducting guidewire inside;

FIG. 7b shows a cross section of an insulated embodiment of the conducting guidewire;

FIG. 8a shows a cross section of a blunt-ended embodiment of the access member with the guidewire inside;

FIG. 8b shows a cross section of a bipolar embodiment of the blunt-ended insulating access member having half-ring termini connected to the opposite poles of the source of electromagnetic energy via imbedded conductive filaments;

FIG. 8c shows a cross section of the bipolar access member embodiment with the terminal conducting half rings shaped in to a cut-off cone;

FIG. 9a shows a source of electromagnetic energy connected to a clamp that may be used to connect the source to an insulated guidewire;

FIG. 9b shows a cross section of the clamp electrically connected to an insulated guidewire by cutting through insulation;

FIG. 9c shows the guidewire stabilized at a predetermined position with respect to the access member with the clamp which is stopped and stabilized by a hub placed at the proximal end of the access member;

FIG. 10a shows a cross section of a prior art hypodermic needle;

FIG. 10b shows a cross section of the hypodermic needle covered with an insulating sheath;

FIG. 10c shows a cross section of a hypodermic needle having an electromagnetic energy conducting guidewire inside;

FIG. 10d shows a cross section of the insulated hypodermic needle having an electromagnetic energy conducting guidewire inside;

FIG. 11a shows a cross section of a hypodermic needle with a guidewire with a distal end complementary to the shape of the distal end of the needle;

FIG. 11b shows a cross section of a hypodermic needle with a guidewire which is equipped with a conductive tip; and

FIG. 11c shows a cross section of the assembly of a hypodermic needle with a guidewire having a physiological saline solution filling the space between the needle and the guidewire.

### DETAILED DESCRIPTION OF THE INVENTION

The current disclosure describes specifically various embodiments of a device, apparatus and a procedure of delivering electromagnetic energy to a vessel tissue or other mammalian tissue which allows creating a substantially round shaped opening on the tissue while minimizing the size of the opening followed by promoting speedy healing of the resulting wound on the tissue by shrinking the collagen in the tissue. The procedure employs a device which utilizes electromagnetic energy to effectuate minimally invasive collagen containing tissue penetration and using the device for opening and closing a vessel or other tissue.

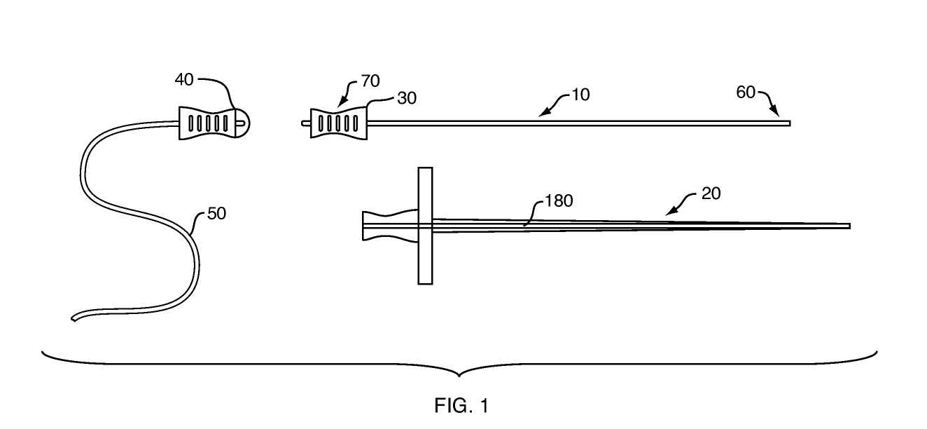

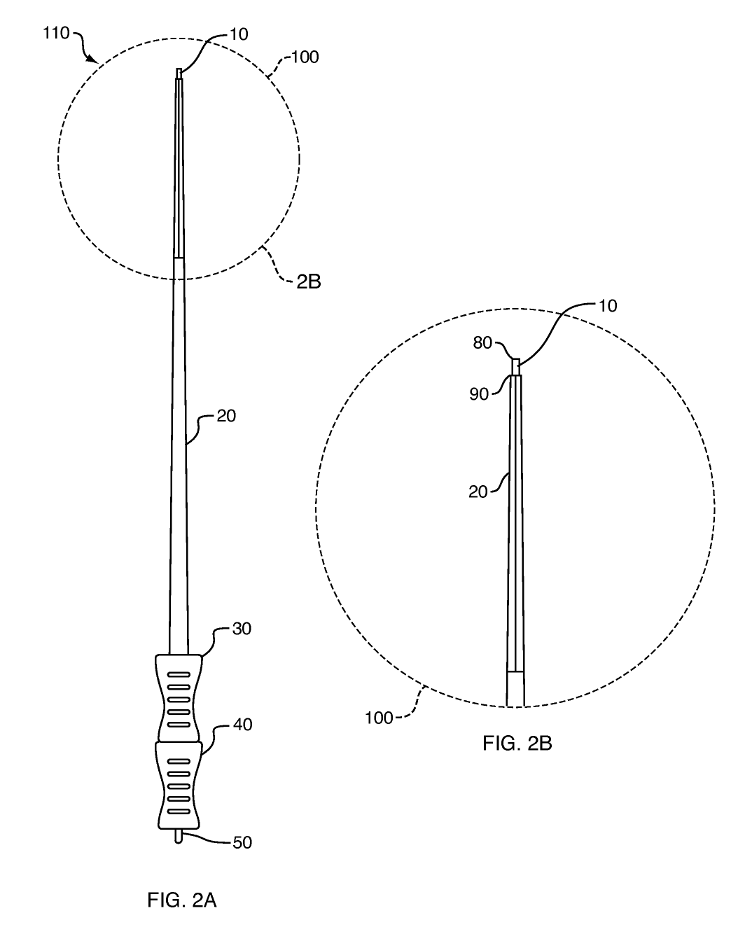

One such embodiment of the device of the invention is illustrated in FIG. 1, FIG. 2a and FIG. 2b. The device comprises a removable access member 10 and a sheath 20 which can encompass the access member 10. The access member 10 has a distal end 60 and a proximal end 70. The distal end 60 of the access member 10 is used for transmitting electromagnetic energy to the tissue, while the proximal end 70 is connected to a source of electromagnetic energy (not shown). Such source can be a source of electricity; heat; infrared, visible, or UV light, ultrasound, etc.

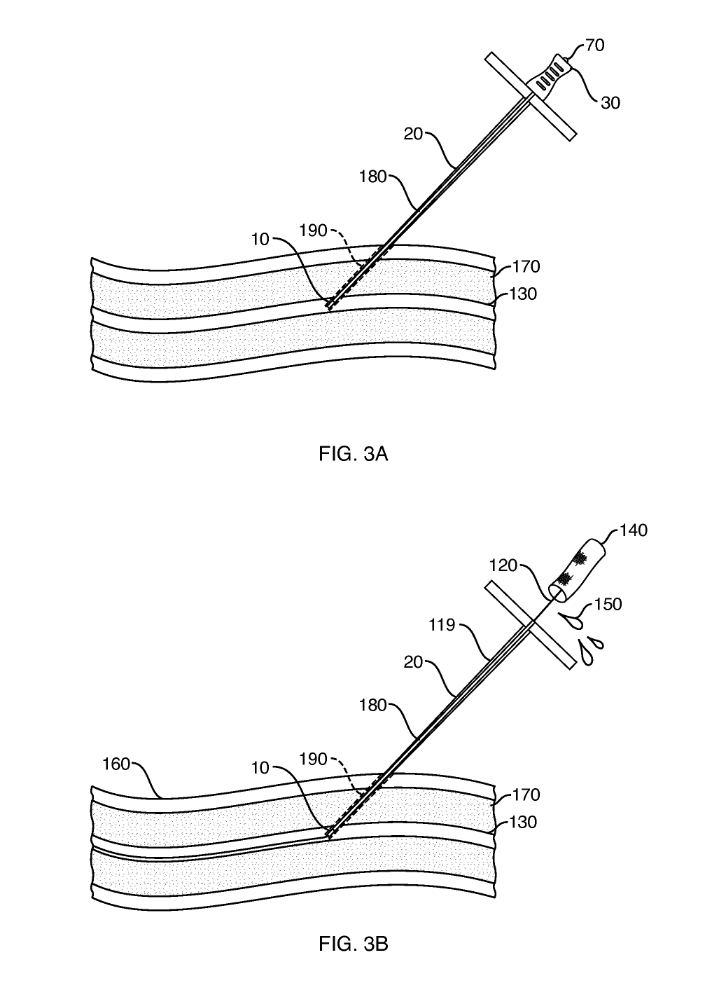

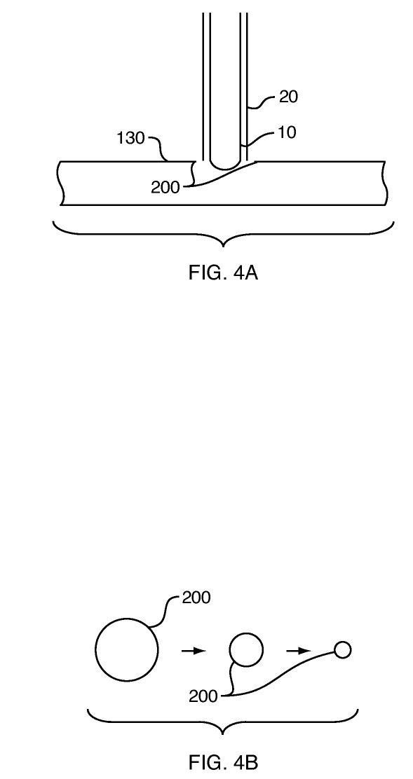

Illustratively, the device utilizes an electric energy source which is connected to the proximal end 70 of the access member 10 via an electrical cord 50 but is not limited hereto. In this embodiment the access member 10 has conductive properties and the sheath 20 has insulating properties. The cord 50 or other transmitting component may be removably connected to the proximal end 70 of the access member 10 with any suitable connector. For example, such a connector can be a luer-type connector with a male luer adapter 30 attached to the proximal end 70 and a matching female adapter 40 attached to the cord 50. The assembled device 110 of the current embodiment is shown in FIG. 2a and FIG. 2b. FIG. 2a shows sheath 20 encompassing access member 10, while FIG. 2b shows an enlarged view of the distal end of the assembly marked with a circle 100 in FIG. 2a. The tip 80 of the access member 10 is positioned with respect to the sheath as to allow transmission of electromagnetic energy to the target tissue. For example, the tip 80 can be slightly protruding beyond the distal edge 90 of the sheath 20, as shown in FIG. 2b. Applying electromagnetic energy allows utilizing a needle-like procedure to penetrate through tissue with a non-sharp device 110 as shown in FIG. 2a and FIG. 2b. The tip 80 of the access member 10 of such a device can be of a blunt shape, as shown in FIG. 2b, with a round cross section but not limited hereto. Utilizing such a tip 80 to penetrate through vessel tissue or other tissue while applying electromagnetic energy to the tissue through the access member 10 results in a vessel opening of a substantially round shape. An illustrative example of using the device 110 of the current invention for accessing inside a vessel is shown in FIG. 3a and FIG. 3b. First the assembly of sheath 20 encompassing access member 10, while applying electromagnetic energy to tissue through access member 10, is used to penetrate through tissue and inside the vessel 130, as shown in FIG. 4a. Subsequently, access member 10 is removed from sheath 20, while leaving sheath 20 in place. Then sheath 20 can be used for accessing inside the vessel 130 or other tissue with, for example, a catheter 140 utilizing a guidewire 120, or with other suitable diagnostic or therapeutic instrument. Control bleed back 150 can be used for positioning the device inside the vessel and for confirming vessel penetration.

One of the embodiments of the method of the current invention can be illustrated with the following example. The access member 10 is placed into the removable sheath 20, for example, but not limited to, 6 French size (2 mm approximate diameter). The sheath 20 insulates the entire access member 10 except the distal tip 60, as shown in FIG. 2a. A power cord 50 which is attached to the proximal end 70 of the access member 10 is plugged into a standard electric source, such as, but not limited to, an operating generator (not shown).

For example, in preparation for applying the assembly 110, user, such as a physician, pulpates the vessel, for example a femoral vessel, to determine puncture location, and positions the assembly 110 accordingly. During the preparation the assembly 110 is less prone to accidentally puncturing the patient or the physician as the tip of the assembly 110 in not sharp. Electromagnetic energy, for example, but not limited to, in the radio frequency range, is then applied to the assembly 110 which is inserted through dermis 160 and subcutaneous tissue 170 approximately 2.5 cm deep, such that it is slightly above the vessel 130. The energy is turned off and the vessel 130 imaging is performed using, but not limited to, a suitable visualization system, such as computer tomography (CT) or ultrasound, or simply confirming that no vessel 130 access has yet occurred by performing a back bleed test 150 by loosening the proximal end 70 connector 30 on the access member 10, which can be a luer connector. No blood should be observed.

Upon completing position verification the used re-applies the energy and inserts the device 110 approximately, but not limited to, 5 mm deeper then turns off the energy. At this point the vessel 130 has been accessed which can be confirmed by CT, ultrasound or a simple back bleed test 150 in which a spurt of blood should be observed (see FIG. 3b). Utilizing this energy off and on method allows the user greater axial depth control so that the vessel is not pierced all the way through. In the latter case repositioning to another site would be required which is not desirable and time consuming.

After the access of the vessel 130 has been accomplished the user withdraws the access member 10 while leaving the sheath 20 in place. Loosening the luer adapter 30 and disconnecting the access member 10 from the source of energy can be used, for example, to facilitate the removal. A component, such as a guidewire 120, is then introduced through the bore 180 of the sheath 20 into the vessel 130, also a length of such component can be introduced into the lumen of the vessel. The guidewire 120, or other component, is left in place to assist in vessel access during subsequent procedures. The sheath 20 facilitates passage of a variety of diagnostic and therapeutic instruments and devices into the vessel 130 and its tributaries. The method and the device of the current invention eliminates the need for a step of inserting the sheath after the guidewire. The method and the device of the current invention prevents excessive bleeding and leaving behind any foreign materials in the patient's body.

At the end of the diagnostic and/or therapeutic procedures the catheters, wires, etc. are removed from the sheath 20 but the sheath 20 is still left in place. The access member 10 is then reintroduced into the sheath 20 so that its distal tip is at the entry of wound 200 of the vessel 130, as shown in FIG. 4a. Electromagnetic energy is then applied through the access member 10 while still within sheath 20. With the access member 10 in place, remodeling of the collagen in the vessel 130 wall leads to the reduction in size of the diameter of the wound 200 (see FIG. 4b). Subsequently, the user retracts the sheath 20 with the access member 10. This procedure provides reduced back bleed due to the electromagnetic energy effect on the access tract 190 (FIG. 3a) where the tissue cells around the circumference and full depth have been micro-cauterized. The latter effect leads to speedy hemostasis which reduces healing times compared to existing procedures.

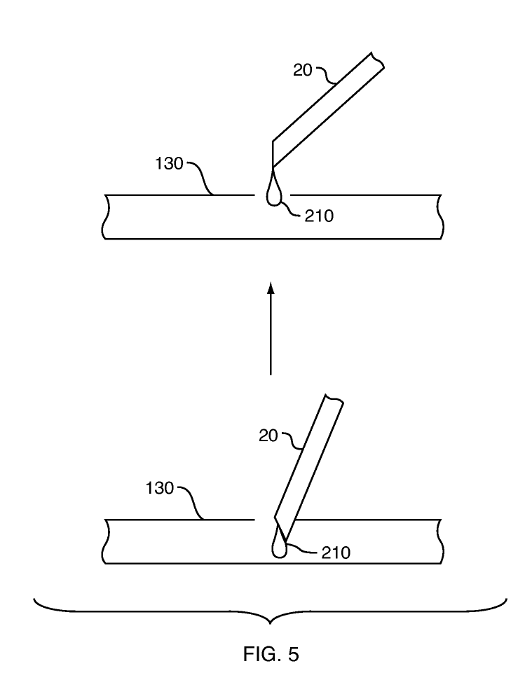

Alternatively, the space 119 between the guidewire 120 and the sheath 20, as shown in FIG. 3b, can be filled with a saline solution. A saline bleb 210 can be held in place at the distal end of the sheath 20 as the sheath 20 is withdrawn, as shown in FIG. 5. To optimize the location of the bleb an ultrasound, a fluoro contrast procedure, or another similar procedure can be used. Saline inside the sheath 20 can serve as the conductive media for transmitting electromagnetic energy from the generator to the bleb 210.

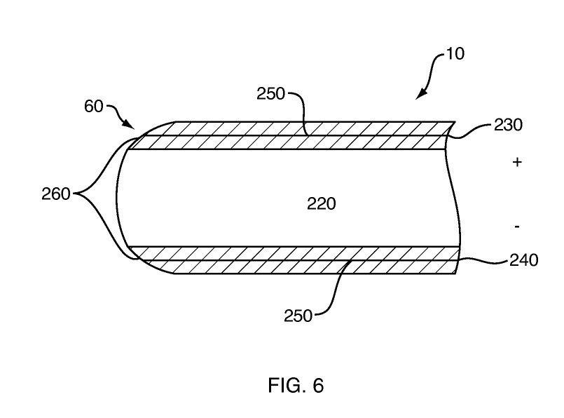

Another embodiment of the device for penetrating tissue utilizing electromagnetic energy is shown in FIG. 6. This embodiment utilizes a bipolar conductive access member 10 having substantially rounded distal end 60 edges and a hollow channel 220 inside. The embodiment does not utilize a sheath. The opposite conductive sides 230 and 240 of the access member 10 are insulated from each other and are connected to the opposite poles of the source of electromagnetic energy. This trim access member 10 can have exposed conductive lead edges. The exposed conductive edges can be made by coextruding fine conductive filaments 250 (e.g. thin wires) in otherwise non-conductive walls of the access member 10 (which can be, but is not limited to, a tube or a catheter) ensuring that the distal ends 260 of the filaments 250 are exposed and conduct electromagnetic energy into tissue when in contact with the tissue. Alternatively, a conductive bulb can be placed at the distal end 60, as describe earlier.

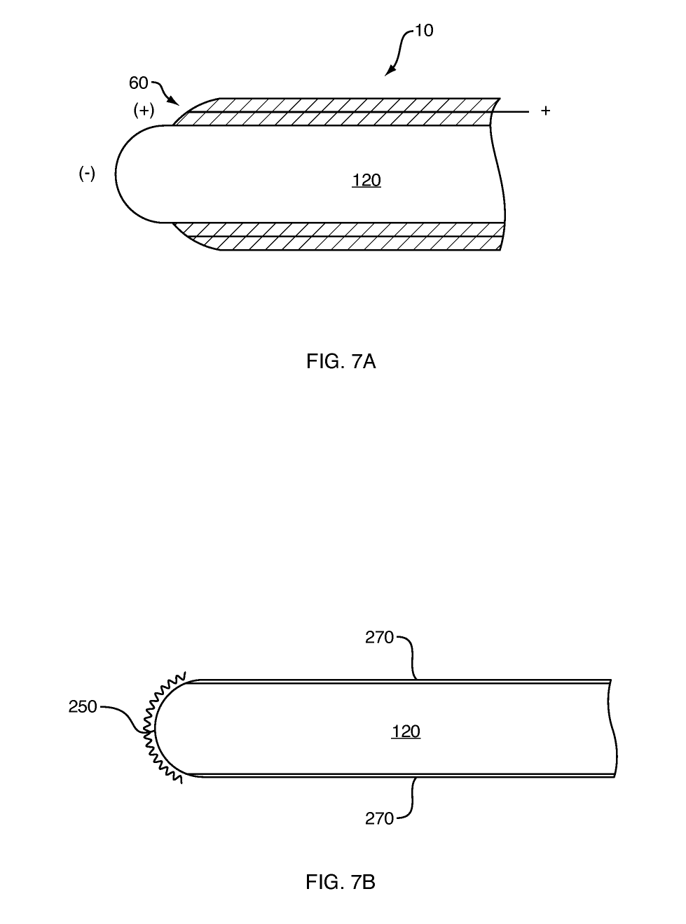

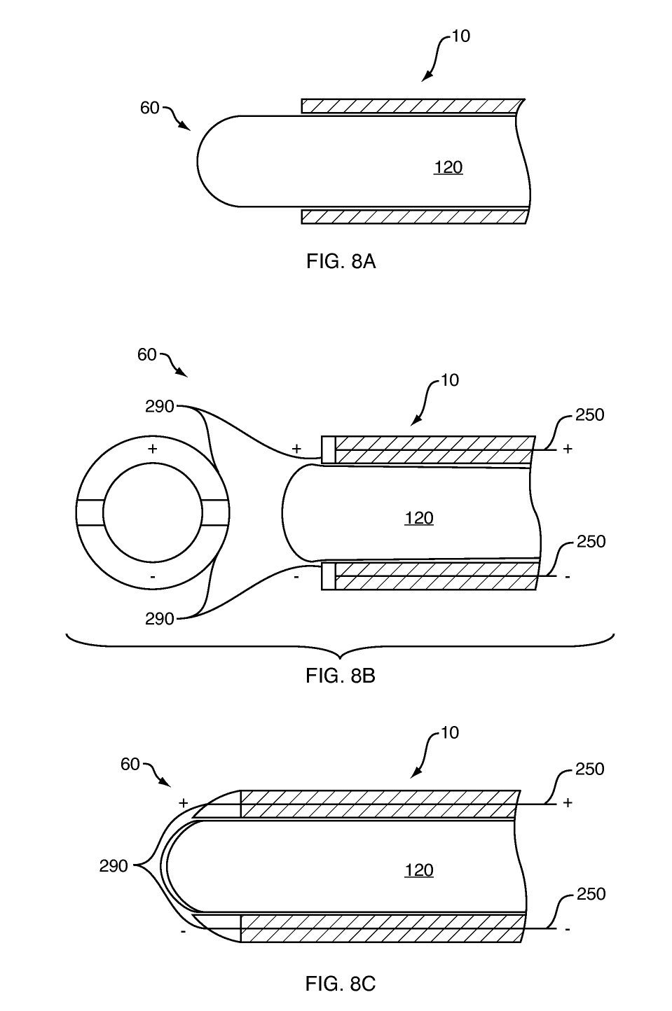

Yet another embodiment of the device of the current invention is shown in FIGS. 7a and 7b. In this embodiment a combination of a conductive access member 10 with a conductive guidewire 120 is utilized, which access member 10 and the guidewire 120 are connected to the opposite poles of a source of electromagnetic energy, as shown in FIG. 7a. The surface of the guidewire 120 of the present embodiment can be insulated substantially in its entirety, as indicated in FIG. 7b with thick solid lines 270, leaving only the surface of its distal end 280 exposed, as shown in FIG. 7b. Utilizing electromagnetic energy to augment tissue penetration allows using access members 10 having substantially blunt distal ends 60, as shown in FIG. 8a. Such an access member 10 may be made of a non-conductive material but having conductive filaments 250 imbedded into its walls for conducting energy to its distal end 60 which can terminate with edges made of a conductive matter 290, as shown in FIG. 8b. This Figure shows a bipolar access member 10 terminating with conductive half rings 290 which are insulated from each other. The half rings can be shaped as to create a cut-off cone shaped distal edge 60 of the access member 10, as shown in FIG. 8c.

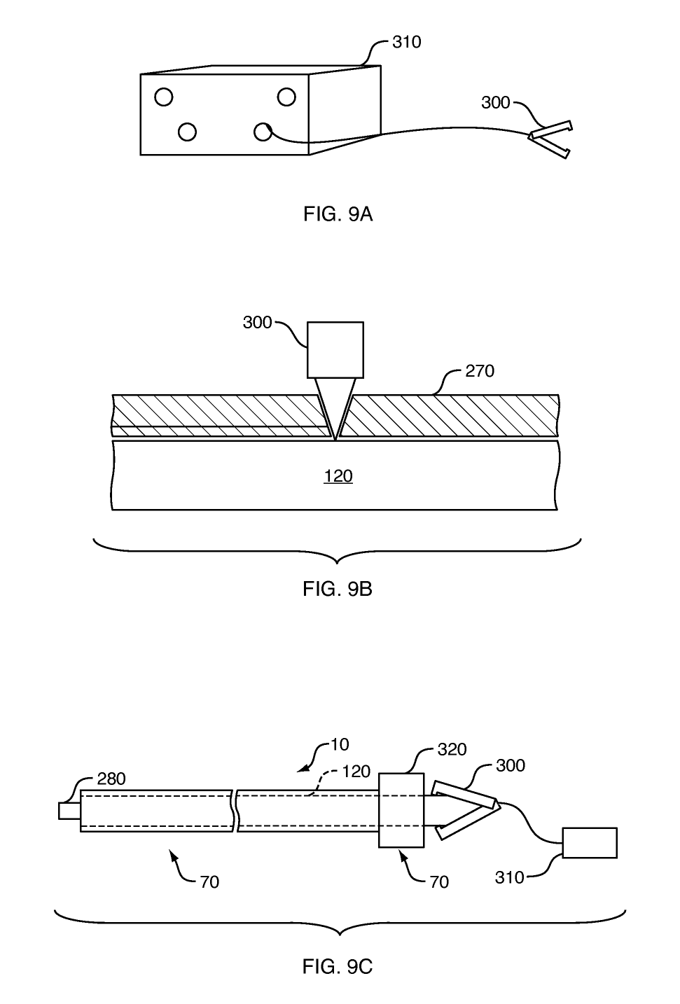

A clamp connector 300 can be used for connecting the guidewire 120 to the energy source 310, as shown in FIG. 9a. The clamp cuts through the insulation 270 on the guidewire 120 and establishes a conductive connection with the guidewire 120, as shown in FIG. 9b. The clamp 300 can also serve a stopper-stabilizer function by not allowing the distal end 280 of the guidewire 120 to protrude beyond a predefined distance from the distal end 60 of the access member 10 and by stabilizing the guidewire 120 in place, as shown in FIG. 9c. A hub 320 can be placed at the proximal end 70 of the access member 10 to facilitate the stopping and the stabilization of the guidewire 120 to which clamp 300 is attached.

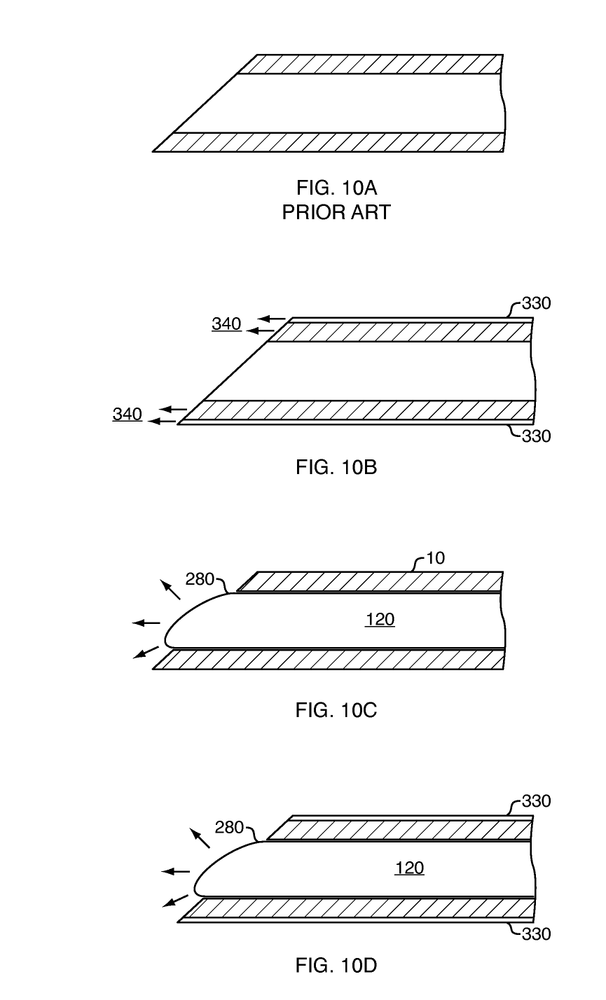

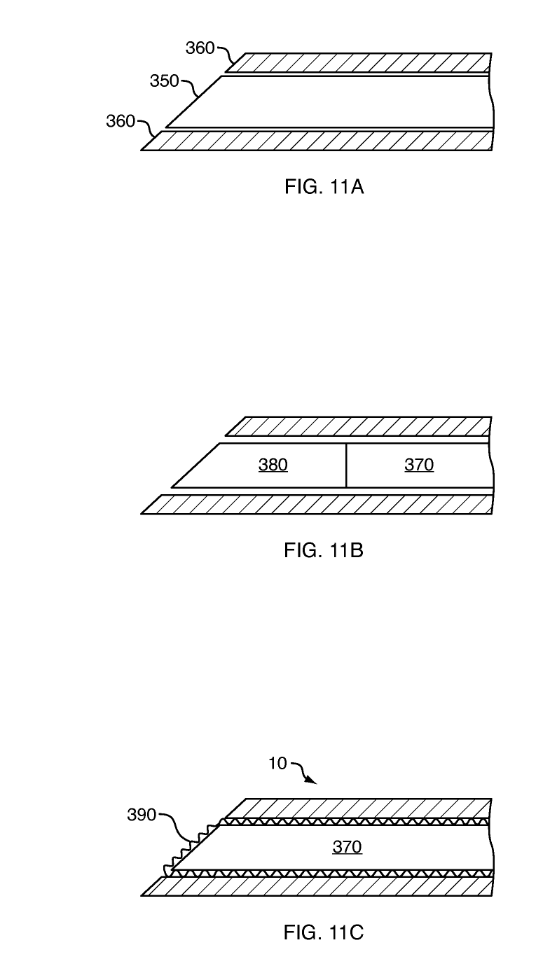

Still other embodiments of the device of the current invention is illustrated in FIG. 10(a-d) and FIG. 11(a-c). These embodiments utilize a conventional hypodermic needle, shown in FIG. 10a, as the conductive access member 10. The outer surface of the needle can be insulated with a removable sheath layer, as shown with solid thick lines 330 in FIG. 10b, to promote distal only energy transfer 340 to the tissue. The sheath can be removed, for examples, by peeling away. A guidewire 120 having a round distal end 280 can be used in combination with the access member 10 of the current embodiment, as shown in FIG. 10c and FIG. 10d and as disclosed earlier. To promote easier tissue penetration the distal end of the guidewire of the present embodiment can be shaped to be complementary to the shape 350 of the distal end of the hypodermic needle 360, as shown in FIG. 11a. A conventional non-conductive guidewire 370 can be equipped with a conductive tip 380 to be used in the current embodiment, as shown in FIG. 11b. As an alternative means for conducting energy to the tip of the guidewire 370 the standard physiological saline solution 390 can be used when placed into the space between the access member 10 and the guidewire 370, as shown in FIG. 11c.

Although the invention has been described with respect to various embodiments, it should be realized that this invention is also capable of a wide variety of further and other embodiments within the spirit of the invention.

What is claimed is:

1. A device for penetrating tissue, the device comprising:
a removable access member having a distal end and a proximal end, said proximal end being connectable to a source of electromagnetic energy; and
a sheath which encompasses said access member while allowing connection of said proximal end of said access member to the source of electromagnetic energy in order to permit the energy to be transmitted to the tissue via said distal end;
wherein penetration of the tissue and withdrawal from the tissue are facilitated by the use of the electromagnetic energy.

2. The tissue penetrating device of claim 1, in which said access member is electrically conductive.

3. The tissue penetrating device of claim 2, in which said sheath has insulating properties.

4. The tissue penetrating device of claim 1, in which said proximal end of said access member includes a connective adapter.

5. The tissue penetrating device of claim 4, in which said connective adapter is a lure adapter.

6. The tissue penetrating device of claim 4, in which said connective adapter is an electric connector.

7. The tissue penetrating device of claim 1, further comprising a source of electromagnetic energy connected to said access member.

8. A method for penetrating a tissue in order to perform a medical procedure, said method comprising:
creating an access tract through the tissue while applying electromagnetic energy with a device having a removable access member and a sheath encompassing said access member;
withdrawing said access member while leaving in place said sheath;
performing a medical procedure; and
withdrawing said sheath.

9. The method of claim 8, further comprising reintroducing said access member into said sheath and applying electromagnetic energy to said access member prior to withdrawing said sheath.

10. The method of claim 8, further comprising positioning a distal end of said access member and said sheath of said device within said tissue tract at a predetermined distance proximal to a vessel.

11. The method of claim 8, further comprising utilizing said sheath for accessing into said tissue of a diagnostic or a therapeutic instrument or device.

12. The method of claim 8, in which said electromagnetic energy is selected from a group consisting of electricity, ultrasound, and light.

13. The method of claim 10, in which said access device is substantially centered with respect to the vessel circumference and outside of said circumference and the energy is applied.

14. The method of claim 13, in which the access device is positions at a distance about 1.5 cm away from said circumference.

15. The method of claim 13, in which computer tomography, ultrasound or manual bleed back positioning tests are performed.

16. The method of claim 15, further comprising entering the vessel at a pre-determined depth while utilizing said positioning tests.
